(19) United States
(12) Patent Application Publication
(10) Pub. No.: US 2024/0386444 A1
(43) Pub. Date: Nov. 21, 2024

(54) METHODS, SYSTEMS, AND COMPUTER PROGRAM PRODUCT FOR VALIDATING A DRUG PRODUCT WHILE BEING HELD BY A DRUG PRODUCT PACKAGING SYSTEM PRIOR TO PACKAGING

(71) Applicant: Synergie Medicale BRG Inc., Longueuil (CA)

(72) Inventors: Rostom Mabrouk, Boucherville (CA); Abderrahmane Chalah, L'Île-Bizard (CA); Stéphan Lessard, Beloeil (CA); Aritro Biswas, New Town (IN); Goutam Kumar Jha, Hooghly (IN); Nagalapelli Prithvi Raju, Sangareddy (IN)

(21) Appl. No.: 18/636,324

(22) Filed: Apr. 16, 2024

Related U.S. Application Data
(60) Provisional application No. 63/496,454, filed on Apr. 17, 2023.

(51) Int. Cl. G06Q 30/018

(52) U.S. Cl. CPC G06Q 30/018

(57) ABSTRACT

A method includes receiving an image of a drug product held by a drug product package filling system; determining whether the drug product matches an intact profile or a defective profile based on the image using a first Artificial Intelligence (AI) system; and determining a type of the drug product based on the image using a second AI system.

### CROSS-REFERENCE TO RELATED APPLICATION

The present application claims priority from and the benefit of U.S. Provisional Patent Application No. 63/496,454, filed Apr. 17, 2023, in the United States Patent and Trademark Office. The disclosure of which is hereby incorporated herein by reference in its entirety.

### BACKGROUND

The present disclosure relates generally to the packaging of drug products, and, in particular, to methods, systems, and computer program products for validating the integrity and type of a drug product.

Drug product packaging systems may be used in facilities, such as pharmacies, hospitals, long term care facilities, and the like to dispense medications to fill prescriptions. These drug product packaging systems may include systems designed to package medications in various container types including, but not limited to, pouches, vials, bottles, blistercard, and strip packaging. Strip packaging is a type of packaging wherein medications are packaged in individual pouches for administration on a specific date and, in some cases, at a specific time. Typically, individual pouches are removably joined together and often provided in rolls. The pouches can be separated from the roll when needed.

The contents of a drug product package are typically validated once the drug product is placed in the package. Validation of the drug product once inside the package; however, may be more difficult due to the overlap of the drug product in the package, the nature of the packaging exterior, e.g., the level of transparency of the exterior, and/or any printing that may be contained on the packaging exterior.

### SUMMARY

In some embodiments of the inventive concept, a method comprises, receiving an image of a drug product held by a drug product package filling system; determining whether the drug product matches an intact profile or a defective profile based on the image using a first Artificial Intelligence (AI) system; and determining, when the drug product matches the intact profile, a type of the drug product based on the image using a second AI system.

In other embodiments, the method further comprises normalizing the image to account for the position of the drug product on the drug product package filling system.

In still other embodiments, the drug product package filling system comprises a plurality of fingers and the drug product is held on one of the plurality of fingers.

In still other embodiments, the drug product is held on one of the plurality of fingers using suction.

In still other embodiments, determining whether the drug product matches the intact profile or the defective profile based on the image comprises determining whether the drug product matches the intact profile or the defective profile based on the normalized image using the first AI system; and determining the type of the drug product based on the image comprises determining the type of the drug product based on the normalized image using the second AI system.

In still other embodiments, the method further comprises: generating embeddings for a plurality of features of the drug product, respectively, using the second AI system; and determining a similarity between the embeddings for the plurality of features of the drug product and feature embeddings for a plurality of drug product types; wherein determining the type of the drug product comprises determining the type of the drug product based on the similarity between the embeddings for the plurality of features of the drug product and the feature embeddings for the plurality of drug product types.

In still other embodiments, the plurality of features of the drug product comprise drug product shape, drug product size, drug product color, an etching on the drug product, an imprint on the drug product, an area of the drug product, and/or a label on the drug product.

In still other embodiments, the plurality of drug product types comprises a plurality of drug product names and/or a plurality of National Drug Code (NDC) identifiers.

In still other embodiments, the method further comprises: augmenting data associated with one or more of a plurality of features of the drug product; and generating embeddings for the plurality of features of the drug product, respectively, using the first AI system responsive to augmenting the data associated with the one or more of the plurality of features of the drug product.

In still other embodiments, the plurality of features of the drug product comprises drug product shape, drug product size, drug product color, an etching on the drug product, an imprint on the drug product, an area of the drug product, a label on the drug product, cracks in the drug product, uneven surfaces of the drug product, chips in the drug product surface, color deviations in the drug product, shape deviations in the drug product, lamination of the drug product, irregular edges of the drug product, dents in the drug product, splits in the drug product, joints in the seams in the drug product, residue on the drug product, deformation of the drug product, and/or bubbles inside the drug product; wherein the first AI system comprises a plurality of neural network models, the plurality of neural network models differing from each other with respect to node weights and/or activation functions; and wherein the method further comprises: determining, using each of the plurality of neural network models, a similarity between the embeddings for the plurality of features of the drug product and feature embeddings for a plurality of intact and defective drug product types, respectively; wherein determining whether the drug product matches the intact profile or the defective profile comprises determining, using each of the plurality of neural network models, whether the drug product matches the intact profile or the defective profile based on the similarity between the embeddings for the plurality of features of the drug product and feature embeddings for a plurality of intact and defective drug product types, respectively.

In still other embodiments, at least one of the plurality of neural network models comprises an AI framework different than others of the plurality of neural network models.

In still other embodiments, the first AI system further comprises a K nearest neighbor neural network model; wherein the method further comprises: generating embeddings for a plurality of features of the drug product, respectively, using the K nearest neighbor neural network model; and determining a similarity between the embeddings for the plurality of features of the drug product and the feature embeddings for the plurality of intact and defective drug product types, respectively; wherein determining whether the drug product matches the intact profile or the defective profile comprises determining whether the drug product matches the intact profile or the defective profile based on a number of the K most similar feature embeddings of the plurality of intact and defective drug product types that are intact and a number of the K most similar feature embeddings of the plurality of intact and defective drug product types that are defective.

In still other embodiments, the method further comprises: aggregating the determinations of the plurality of neural network models and the K nearest neighbor network model on whether the drug product matches the intact profile or the defective profile; and determining whether the drug product matches the intact profile or the defective profile based on the aggregation of the determinations of the plurality of neural network models and the K nearest neighbor network model.

In some embodiments of the inventive concept, a system comprises: a processor; and a memory coupled to the processor and comprising computer readable program code embodied in the memory that is executable by the processor to perform operations comprising: receiving an image of a drug product held by a drug product package filling system; determining whether the drug product matches an intact profile or a defective profile based on the image using a first Artificial Intelligence (AI) system; and determining, when the drug product matches the intact profile, a type of the drug product based on the image using a second AI system.

In further embodiments, the operations further comprise: normalizing the image to account for the position of the drug product on the drug product package filling system; wherein determining whether the drug product matches the intact profile or the defective profile based on the image comprises determining whether the drug product matches the intact profile or the defective profile based on the normalized image using the first AI system; and wherein determining the type of the drug product based on the image comprises determining the type of the drug product based on the normalized image using the second AI system.

In still further embodiments, the operations further comprise: generating embeddings for a plurality of features of the drug product, respectively, using the second AI system; and determining a similarity between the embeddings for the plurality of features of the drug product and feature embeddings for a plurality of drug product types; wherein determining the type of the drug product comprises determining the type of the drug product based on the similarity between the embeddings for the plurality of features of the drug product and the feature embeddings for the plurality of drug product types.

In still further embodiments, the operations further comprise: augmenting data associated with one or more of a plurality of features of the drug product; and generating embeddings for the plurality of features of the drug product, respectively, using the first AI system responsive to augmenting the data associated with the one or more of the plurality of features of the drug product.

In still further embodiments, the plurality of features of the drug product comprises drug product shape, drug product size, drug product color, an etching on the drug product, an imprint on the drug product, an area of the drug product, a label on the drug product, cracks in the drug product, uneven surfaces of the drug product, chips in the drug product surface, color deviations in the drug product, shape deviations in the drug product, lamination of the drug product, irregular edges of the drug product, dents in the drug product, splits in the drug product, joints in the seams in the drug product, residue on the drug product, deformation of the drug product, and/or bubbles inside the drug product; wherein the first AI system comprises a plurality of neural network models, the plurality of neural network models differing from each other with respect to node weights and/or activation functions; and wherein the operations further comprise: determining, using each of the plurality of neural network models, a similarity between the embeddings for the plurality of features of the drug product and feature embeddings for a plurality of intact and defective drug product types, respectively; wherein determining whether the drug product matches the intact profile or the defective profile comprises determining, using each of the plurality of neural network models, whether the drug product matches the intact profile or the defective profile based on the similarity between the embeddings for the plurality of features of the drug product and feature embeddings for a plurality of intact and defective drug product types, respectively.

In still further embodiments, the first AI system further comprises a K nearest neighbor neural network model; wherein the method further comprises: generating embeddings for a plurality of features of the drug product, respectively, using the K nearest neighbor neural network model; and determining a similarity between the embeddings for the plurality of features of the drug product and the feature embeddings for the plurality of intact and defective drug product types, respectively; wherein determining whether the drug product matches the intact profile or the defective profile comprises determining whether the drug product matches the intact profile or the defective profile based on a number of the K most similar feature embeddings of the plurality of intact and defective drug product types that are intact and a number of the K most similar feature embeddings of the plurality of intact and defective drug product types that are defective.

In some embodiments of the inventive concept, a computer program product comprises: a non-transitory computer readable storage medium comprising computer readable program code embodied in the medium that is executable by a processor to perform operations comprising: receiving an image of a drug product held by a drug product package filling system; determining whether the drug product matches an intact profile or a defective profile based on the image using a first Artificial Intelligence (AI) system; and determining, when the drug product matches the intact profile, a type of the drug product based on the image using a second AI system.

Other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter, and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including an Artificial Intelligence (AI) assisted drug product validation system in accordance with some embodiments of the inventive concept;

FIG. 2 is an elevation view of the drug product packaging system fingers that are used to move drug product between containers and a package in accordance with some embodiments of the inventive concept;

FIG. 3 is a perspective view of cameras configured to capture images of drug product held by the drug product packaging system fingers in accordance with some embodiments of the inventive concept;

FIG. 4 is a block diagram of a drug product validation system in accordance with some embodiments of the inventive concept;

FIG. 5 is a block diagram of a neural network architecture for use in the AI models of FIGS. 1 and 4;

FIG. 6 is a block diagram of the AI models used for validating the integrity of a drug product in accordance with some embodiments of the inventive concept;

FIGS. 7-9 are flowcharts that illustrate operations of a drug product validation system according to some embodiments of the inventive concept;

FIG. 10 is a data processing system that may be used to implement one or more servers for performing drug product validation in accordance with some embodiments of the inventive concept; and

FIG. 11 is a block diagram that illustrates a software/hardware architecture for use in the AI assisted drug product validation system of FIG. 1 in accordance with some embodiments of the inventive concept.

FIG. 12 is a diagram of a mobile calibration cassette in accordance with some embodiments of the inventive concept.

### DETAILED DESCRIPTION

In the following detailed description, numerous specific details are set forth to provide a thorough understanding of embodiments of the present inventive concept. However, it will be understood by those skilled in the art that the present invention may be practiced without these specific details. In some instances, well-known methods, procedures, components and circuits have not been described in detail so as not to obscure the present inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “data processing facility” includes, but it is not limited to, a hardware element, firmware component, and/or software component. A data processing system may be configured with one or more data processing facilities.

The term “drug product packaging system,” as used herein, refers to any type of pharmaceutical dispensing system including, but not limited to, automated systems that fill vials, bottles, containers, pouches, blistercards, or the like with drug product, semi-automated systems that fill vials, bottles, containers, pouches, blistercards, or the like with drug product, and any combination of automated and semi-automated systems for filling a drug product package with drug product. Drug product packaging system also includes packaging systems for pharmaceutical alternatives, such as nutraceuticals and/or bioceuticals.

The terms “pharmaceutical” and “medication,” as used herein, are interchangeable and refer to medicaments prescribed to patients either human or animal. A pharmaceutical or medication may be embodied in a variety of ways including, but not limited to, pill form capsule form, tablet form, and the like.

The term “drug product” refers to any type of medicament that can be packaged within a vial, bottle, container, pouch, blistercard, or the like by automated and semi-automated drug product packaging systems including, but not limited to, pills, capsules, tablets, caplets, gel caps, lozenges, and the like. Drug product also refers to pharmaceutical alternatives, such as nutraceuticals and/or bioceuticals. Example drug product packaging systems including management techniques for fulfilling packaging orders are described in U.S. Pat. No. 10,492,987 the disclosure of which is hereby incorporated herein by reference.

The term “drug product package” refers to any type of object that can hold a drug product including, but not limited to, a vial, bottle, container, pouch, blistercard, or the like.

Embodiments of the inventive concept are described herein in the context of a drug product validation engine for validating a drug product before it is placed inside a package. The drug product validation engine may include multiple Artificial Intelligence (AI) engines, which use multi-layer neural network technology. The embodiments of the system for validating drug product prior to packaging may, therefore, be described with respect to the use of one or more multi-layer neural network systems. It will be understood, however, that embodiments of the inventive concept are not limited to multi-layer neural network implementations of the drug product validation system and that other types of AI systems may be used including, but not limited to, a machine learning system, a deep learning system, a natural language processing system, and/or computer vision system. Moreover, it will be understood that the multi-layer neural network is a multi-layer artificial neural network comprising artificial neurons or nodes and does not include a biological neural network comprising real biological neurons. The AI systems, such as the multi-layer neural networks described herein, may be configured to transform a memory of a computer system to include one or more data structures, such as, but not limited to, arrays, extensible arrays, linked lists, binary trees, balanced trees, heaps, stacks, and/or queues. These data structures can be configured or modified through the AI training process to improve the efficiency of a computer system when the computer system operates in an inference mode to make an inference, prediction, classification, suggestion, or the like with respect to validating a drug product prior to packaging with respect to the integrity of the drug product, e.g., whether the drug product is intact or defective, and/or with respect to the type of the drug product, e.g., the name or National Drug Code (NDC)/Drug Identification Number (DIN) identifier, in response to input information or data provided thereto. It will be understood that the type of drug product can be identified using any type of nomenclature for the drug type including name, NDC, DIN, or other identifier in accordance with different embodiments of the inventive concept.

Some embodiments of the inventive concept stem from a realization that validating the contents of a drug product package, such as a pouch or blistercard, for example, may be difficult due to overlap of drug product in the package, visibility through the packaging exterior, and/or printing or other indicia that may be on the on the packaging exterior. Some embodiments of the inventive concept may provide a drug product validation engine that is configured to validate the integrity of the drug product, e.g., whether the drug product is intact or is defective in some way, and/or determine the type of drug product, e.g., name of the drug product or NDC identifier, prior to the drug product being packaged. The drug product validation engine may use multiple trained AI models, such as multi-layer neural networks, to classify the drug product with respect to being intact or defective and to classify the drug product type based on name or NDC identifier, for example. In some embodiments, multiple trained neural network models may be used to classify whether the drug product is intact or defective. Each of these neural network models may classify the drug product as intact or defective and their classifications may be aggregated to determine a final classification. These neural network models may differ based on the weights and/or activation functions they use at each node in some embodiments. In further embodiments, one or more of the neural network models may comprise an AI framework that is different than the others. For example, one of the neural network models may use the Py Torch framework while others of the neural network models use a different framework. One or more of the neural network models may comprise a K nearest neighbor neural network model that can be used to classify a drug product as defective or intact based on the status, i.e., defective or intact, of its K closest neighbors. The aggregation of the classifications, intact or defective, can be done in a variety of different ways including basing the final classification on a simple majority of the classifications of the different neural networks, setting a threshold for the number of intact or defective classifications that must be met before selecting that classification as the final classification, or a more complex aggregation in which one or more of the neural network classifications, such as the classification based on the K nearest neighbor neural network, is given precedence and only if that neural network classifies the drug product as intact are the other neural network considered as possibly determining that the drug product is defective.

For drug product that is determined to be intact, one or more multi-classification, multi-layer neural networks may be used to classify the drug product based on drug product name and/or other indicia, such as NDC identification and/or Drug Identification Number (DIN). Thus, a drug product validation engine may be used to validate both the integrity of drug product being packaged as well as validate the type of drug product being packaged prior to packaging taking place, which may improve the accuracy of the validation as the validation may be performed without having to account for any obfuscation due to the packaging.

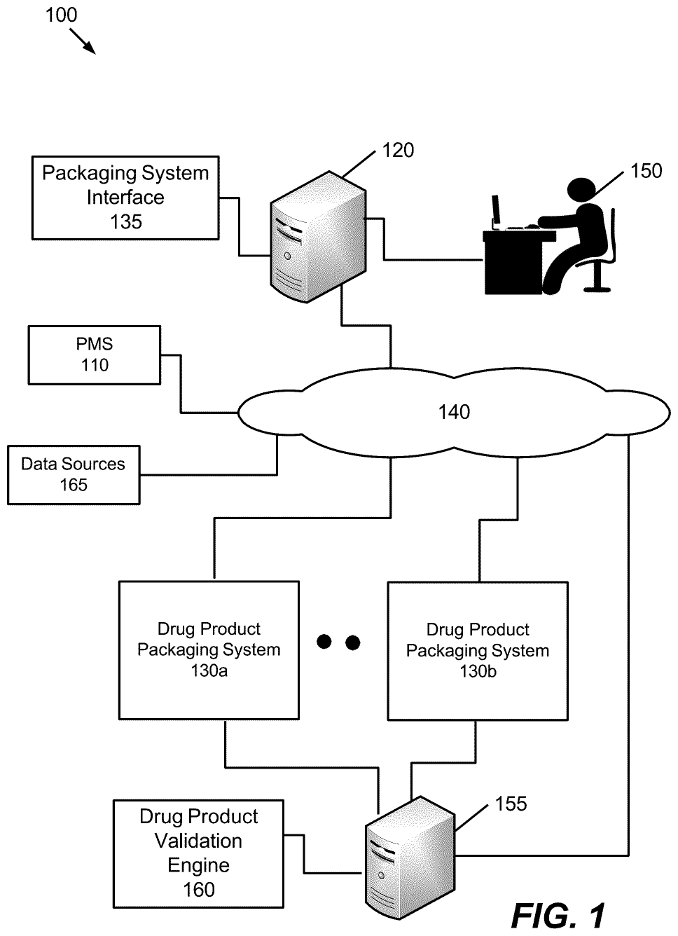

Referring to FIG. 1, a communication network 100 including a drug product validation system, in accordance with some embodiments of the inventive concept, comprises a pharmacy management system (PMS) or host system 110, a packaging system server 120, a validation system server 155, and one or more drug product packaging systems 130a and 130b that are coupled via a network 140 as shown.

The PMS system 110 may be configured to manage and fill prescriptions for customers. As used herein, PMS systems may be used in pharmacies or may be used generally as batch-generating systems for other applications, such as dispensing nutraceuticals or bioceuticals. The PMS system 110 may be associated with a variety of types of facilities, such as pharmacies, hospitals, long term care facilities, and the like. The PMS system or host system 110 may be any system capable of sending a valid prescription to the one or more product packaging systems 130a and 130b. The packaging system server 120 may include a packaging system interface module 135 and may be configured to manage the operation of the drug product packaging systems 130a and 130b. For example, the packaging system server 120 may be configured to receive packaging orders from the PMS system 110 and to identify which of the drug product packaging systems 130a and 130b should be used to package individual orders or batches of orders. In addition, the packaging system server 120 may be configured to manage the operations of the drug product packaging systems 130a and 130b. For example, the packaging system server 120 may be configured to manage the inventory of drug product available through each of the drug product packaging systems 130a and 130b, to manage the drug product dispensing canisters assigned or registered to one or more of the drug product packaging systems 130a and 130b, to manage the operational status generally of the drug product packaging systems 130a and 130b, and/or to manage reports regarding the status (e.g., assignment, completion, etc.) of packaging orders, drug product inventory, order billing, and the like. A user 150, such as a pharmacist or pharmacy technician, may communicate with the packaging system server 120 using any suitable computing device via a wired and/or wireless connection. Although the user 150 is shown communicating with the packaging system server 120 via a direct connection in FIG. 1, it will be understood that the user 150 may communicate with the packaging system server 120 via one or more network connections. The user 150 may interact with the packaging system server 120 to approve or override various recommendations made by the packaging system server 120 in operating the drug product packaging systems 130a and 130b. The user 150 may also communicate with the PMS 110 as prescription orders may be entered manually into the PMS 110. The user 150 may also initiate the running of various reports as described above for the drug product packaging systems 130a and 130b. Although only two drug product packaging systems 130a and 130b are shown in FIG. 1, it will be understood that more than two drug product packaging systems or a single drug product packaging system may be managed by the packaging system server 120.

The drug product validation system may include the validation system server 155, which includes a drug product validation engine module 160 to facilitate validation of drug product while it is held by the drug product packaging system 130a, 130b, but before it is packaged. The validation system server 155 and drug product validation engine module 160 may include multiple trained AI models, such as multi-layer neural networks and accompanying logic that are configured to classify the integrity of the drug product, e.g., whether the drug product is intact or defective, and/or to classify the type of drug product by drug product name or other indicia, such as National Drug Codes (NDCs) or Drug Identification Numbers (DINs). The validation system server 155 may access data sources 165 as part of the drug product validation process. These data sources 165 may include drug product reference sources, such as databases containing National Drug Codes (NDCs) or Drug Identification Numbers (DINs). The data sources 165 may also include packaging data from historical orders, which may be used in drug product integrity evaluations. The data sources 165 may further include characteristic information for drug products, such as capsules, pills, and the like, including size, shape, color, markings, or other identifying information.

It will be understood that the division of functionality described herein between the packaging system server 120/packaging system interface module 135 and the validation system server 155/drug product validation engine module 160 is an example. Various functionality and capabilities can be moved between the packaging system server 120/packaging system interface module 135 and the validation system server 155/drug product validation engine module 160 in accordance with different embodiments of the inventive concept. Moreover, in some embodiments, the packaging system server 120/packaging system interface module 135 and the validation system server 155/drug product validation engine module 160 may be merged as a single logical and/or physical entity.

A network 140 couples the drug product packaging systems 130a and 130b, the PMS system 110, the packaging system server 120, and the validation system server 155 to one another. The network 140 may be a global network, such as the Internet or other publicly accessible network. Various elements of the network 140 may be interconnected by a wide area network, a local area network, an Intranet, and/or other private network, which may not be accessible by the general public. Thus, the communication network 140 may represent a combination of public and private networks or a virtual private network (VPN). The network 140 may be a wireless network, a wireline network, or may be a combination of both wireless and wireline networks.

The drug product validation service for validating drug product prior to packaging provided through the validation system server 155 and package validation engine(s) module 160, in some embodiments, may be implemented as a cloud service. In some embodiments, the drug product package validation service may be implemented as a Representational State Transfer Web Service (RESTful Web service).

Although FIG. 1 illustrates an example communication network that includes a drug product validation system for validating a drug product prior to packaging while still held by a drug product packaging system, it will be understood that embodiments of the inventive subject matter are not limited to such configurations, but are intended to encompass any configuration capable of carrying out the operations described herein.

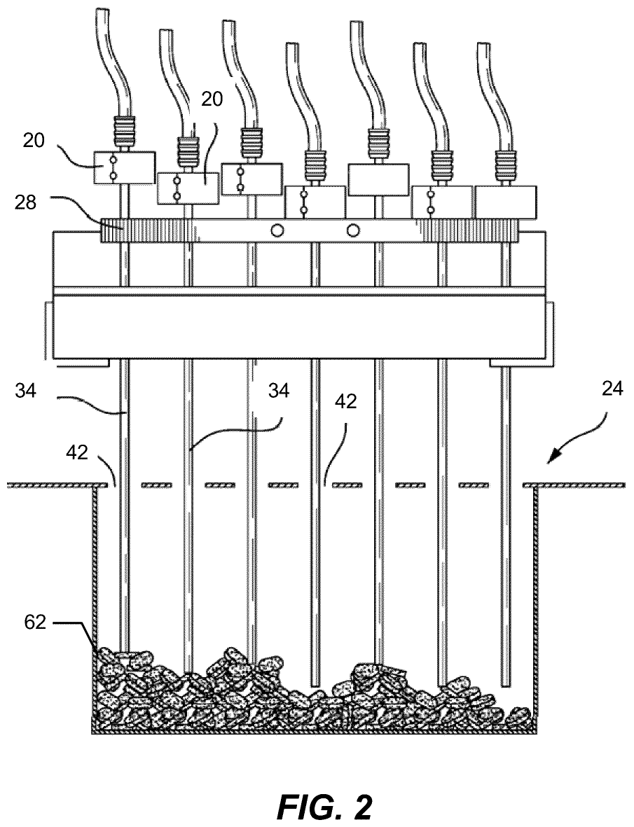

FIG. 2 is an elevation view of a portion of the drug product packaging systems 130a, 130b of FIG. 1 that illustrates the apparatus used to extract drug product from containers for transport to a package for enclosure therein in accordance with some embodiments of the inventive concept. As shown in FIG. 2, the apparatus includes a plurality of fingers or pipettes 34 that are hollow and configured to create a suction force at ends thereof using air flow. Each of the fingers 34 is slidably mounted to a support rack 28 so as to be displaceable in the vertical direction in the elevation view of FIG. 2. A ring 20 is included at a proximate end of each of the fingers 34 to bias the finger 34 downward under the force of gravity. As the amount of drug product 62 in each of the container bins 42 may differ, the fingers 34 may displace in the vertical direction independently allowing each finger to reach the drug product in a respective bin 42 based on the height of the drug product contained in the bin 42. A drug product packaging system or package filling system using fingers as shown in FIG. 2 to retrieve drug product from containers or bins for placement in a drug product package is described, for example, in U.S. Patent Publication No. 2020/0016039, the disclosure of which is hereby incorporated herein by reference.

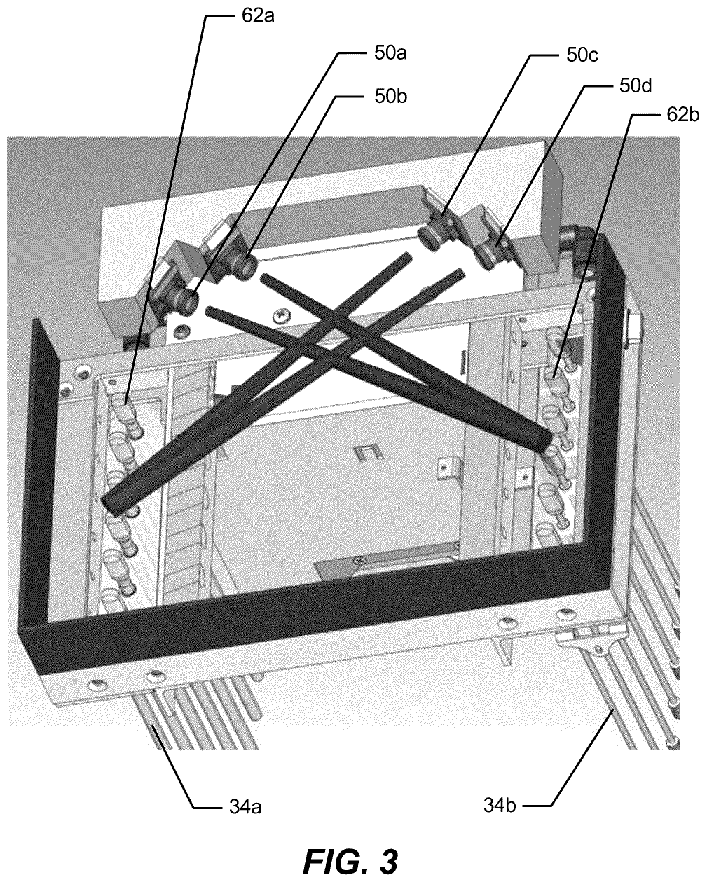

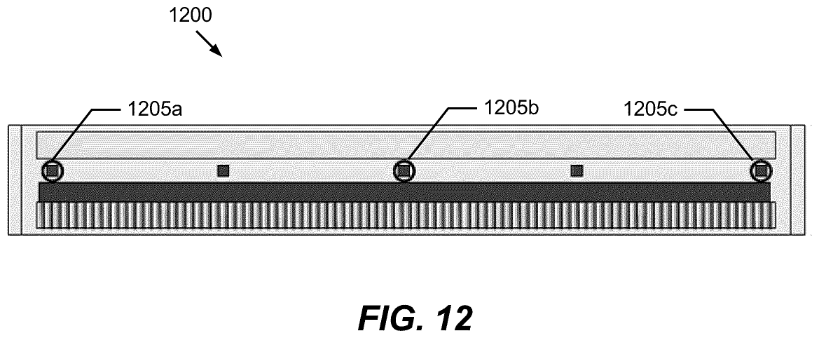

FIG. 3 is a perspective view of cameras configured to capture images of drug product held by the drug product packaging system fingers of FIG. 2 in accordance with some embodiments of the inventive concept. As shown in FIG. 3, the drug product packaging system or package filling system includes two sets of fingers 34a and 34b. Fingers 34a may have a larger diameter for picking up larger drug product 62a elements while fingers 34b may have a smaller diameter for picking up smaller drug product 62b elements. Cameras 50a and 50b may be trained on the fingers 34b to capture images of drug product 62b picked up at the distal ends thereof via suction through the fingers 34b. Similarly, cameras 50c and 50d may be trained on the fingers 34a to capture images of drug product 62a picked up at the distal ends thereof via suction through the fingers 34a. The drug product validation engine 160 of FIG. 1 may include logic to switch between the various cameras 50a, 50b, 50c, and 50d to acquire images of drug product 62a, 62b picked up by the fingers 34a, 34b. Although the example embodiment of FIG. 3 shows two cameras per side 50a, 50b and 50c, 50d, in other embodiments only one camera may be used per side. Due to different distances and angles between the cameras 50a, 50b, 50c, and 50d and the fingers 34a and 34b, the images may be processed to normalize the differences between angle of view and distance to the camera. To ensure that the two or more cameras 50a, 50b, 50c, and 50d capture images of the drug product on the distal ends of the fingers 34a and 34b, the two or more cameras 50a, 50b, 50c, and 50d may be calibrated. As shown in FIG. 12, a mobile calibration cassette 1200 may be installed on one side to calibrate the one or more cameras 50a, 50b, 50c, and 50d on the opposite side. The symbols 1205a, 1205b, and 1205c may be used to adjust the positioning of the cameras. A calibration software tool for the one or more cameras 50a, 50b, 50c, and 50d may be used to provide notification when the position is set. The camera focus may then be adjusted through manual adjustment of the lens until the calibration software tool indicates the image is in focus. The calibration software tool may analyze other camera parameters, such as white balance, RGB balance, and exposure time based on the environment in which drug product packaging system operations. The calibration software may stream a picture with light flashing to adjust polarization. The above-described operations are performed for each camera, i.e., the one or more cameras on each side.

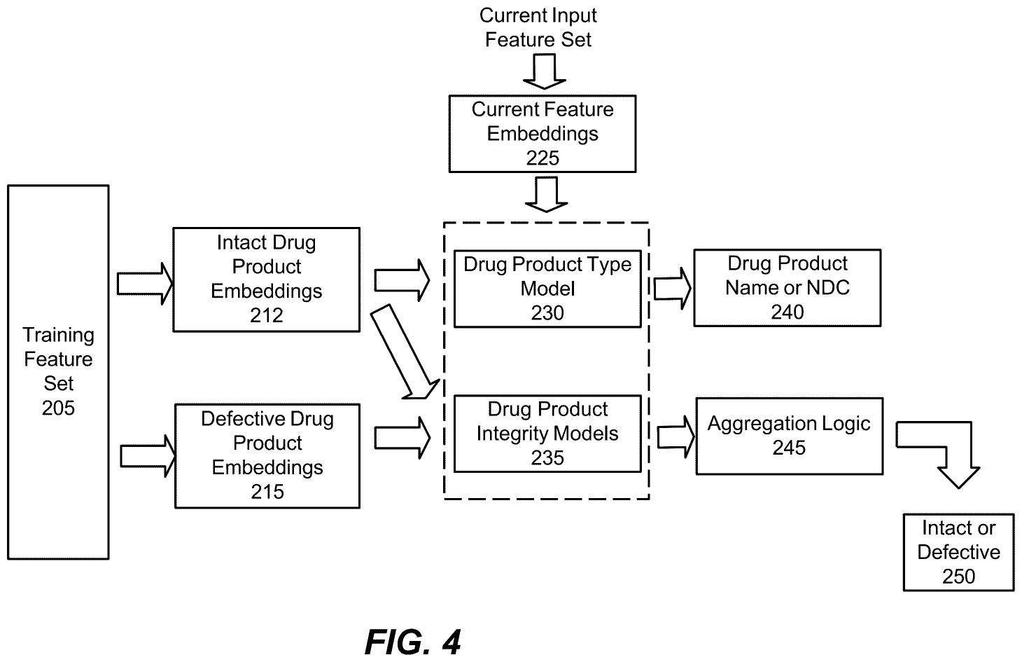

FIG. 4 is a block diagram of a drug product validation system including AI models for classifying the integrity of a drug product and the type of the drug product, e.g., drug product name or other identification, in accordance with some embodiments of the inventive concept. The drug product validation system may be implemented by the validation system server 155 and drug product validation engine module 160 of FIG. 1 according to some embodiments. As shown in FIG. 4, a training feature set 205 for training and/or initializing the various AI algorithms to generate the AI models may include drug product images including images of the drug product held by the drug product packaging system or filling system prior to packaging that contains information associated with a plurality of input variables, which include, but are not limited to, input variables associated with intact drug products and defective drug products. The training feature set 205 may also include information from the data sources 165 that provides details on characteristics of the various drug products to be validated. For example, when drug product is picked up from a container via a finger 34 as described above with respect to FIGS. 2 and 3, the drug product may be a single, intact, drug product element at the end of the finger 34 or may be defective in some way. The types of defects are numerous and can include, but are not limited to, multiple drug product elements being picked up together by the finger 34 or defects in the condition of the drug product. Thus, the input variables may include features corresponding to an intact drug product and features corresponding to a defective drug product. The intact drug product features may include, but are not limited to, one or more of drug product shape, drug product size, drug product color, an etching on the drug product, an imprint on the drug product, an area of the drug product, and a label on the drug product. The defective drug product features may include, but are not limited to, one or more of cracks in the drug product, uneven surfaces of the drug product, chips in the drug product surface, color deviations in the drug product, shape deviations in the drug product, lamination of the drug product, irregular edges of the drug product, dents in the drug product, splits in the drug product, joints in the seams in the drug product, residue on the drug product, deformation of the drug product, and bubbles inside the drug product. Intact drug product embeddings 212 may be generated for the input variables contained in the training feature set 205 that correspond to intact drug product features and defective drug product embeddings 215 may be generated for the input variables contained in the training feature set 205 that correspond to defective drug product features. An embedding is a learned continuous vector representation of a discrete variable. These input variables from the training feature set 205 may be categorical or discrete variables for which a continuous vector is generated. A drug product type multi-layer neural network may be trained using the intact drug product embeddings 212 to generate the drug product type model 230. Similarly, one or more drug product integrity multi-layer neural networks may be trained using both the intact drug product embeddings 212 and the defective drug product embeddings 215 to generate the drug product integrity model(s) 235.

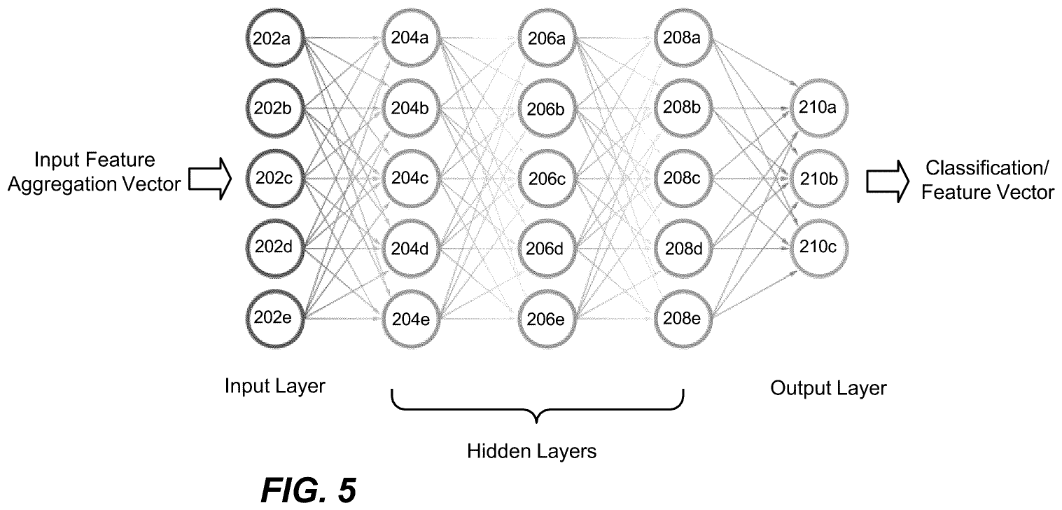

FIG. 5 is a diagram of an example artificial neural network system that can be used to implement the drug product type model 230 and the drug product integrity model(s) 235 of FIG. 4 according to some embodiments of the inventive concept. As shown in FIG. 5, the artificial neural network includes a plurality of node layers comprising an input layer, one or more hidden layers, and an output layer. In the example shown in FIG. 5, an input layer comprises five nodes or neurons 202a, 202b, 202c, 202d, and 202e and an output layer comprises three nodes or neurons 210a, 210b, and 210c. In the example shown, three hidden layers connect the input layer to the output layer including a first hidden layer comprising five nodes or neurons 204a, 204b, 204c, 204d, and 204e, a second hidden layer comprising five nodes or neurons 206a, 206b, 206c, 206d, and 206e, and a third hidden layer comprising five nodes or neurons 208a, 208b, 208c, 208d, and 208e. Other embodiments may use more or fewer hidden layers. Each node or neuron connects to another and has an associated weight and threshold. If the output of any individual node or neuron is above the specified threshold value, that node is activated, sending data to the next layer of the network. Otherwise, no data is passed along to the next layer of the network.

The number of nodes in the input layer 202a, 202b, 202c, 202d, and 202e may correspond to the number of variables in the training feature set 205 used during training and the current input feature set received during inference or classification mode operation. In aggregate, the embedded vectors corresponding to the plurality of input variables may be viewed as a drug product vector. The number of nodes in the output layer 210a, 210b, and 210c may correspond to the number of different classification categories. For each of the drug product integrity models 235, the number of output layer nodes may be two, which correspond to an intact classification and a defective classification, respectively. For each the drug product type model 230, the number of output layer nodes may be numerous corresponding to the different drug product names, NDCs, and/or DINs. In other embodiments, the output layer may generate a feature vector simplifying the image information. In addition, while the drug product integrity model 235 may be configured to distinguish between intact and defective drug products according to some embodiments, in other embodiments, the drug product integrity model 235 may distinguish between other packaging irregularities, such as doubles. For example, the drug product integrity model 235 may be trained to distinguish between extra drug product instances (e.g., doubles) and broken drug product.

As described above, an artificial neural network relies on training data to learn and improve its accuracy over time. The neural network for drug product type model 230 and the neural networks for the drug product integrity models 235 may be trained with numerous images of both intact and defective drug product held by a drug product packaging system, such as at the distal end of the fingers 34 of FIG. 2 along with other textual information and images obtained from drug product reference information repositories represented by data sources 165 in FIG. 1. The neural networks making up the drug product integrity models 235 may be trained to distinguish between intact and defective drug products and the neural network making up the drug product type model 230 may be trained to identify drug products by drug product name or other indicia, such as NDC or DIN. Once the various parameters of the neural network systems are tuned and refined for accuracy, they can be trained to accept images of a drug product held by a drug product packaging system prior to packaging and classify the drug product as being either intact or defective and, when the drug product is classified as being intact, suggest an identification for the drug product by drug product name or other indicia, such as NDC or DIN.

Each individual node or neuron in the artificial neural network may be viewed as implementing a linear regression model, which is composed of input data, weights, a bias (or threshold), and an output. Once an input layer is determined, weights are assigned. These weights help determine the importance of any given variable, with larger ones contributing more significantly to the output compared to other inputs. All inputs are then multiplied by their respective weights and then summed, i.e., a MAC operation. In FIG. 5, node or neuron 206a, for example, receives inputs corresponding to the outputs of nodes or neurons 204a, 204b, 204c, 204d, and 204e. These inputs are multiplied by their corresponding weights and summed at node or neuron 206a. Afterward, the output is passed through an activation function, which determines the output. If that output exceeds a given threshold, it activates the node by passing data to the next layer in the network. This results in the output of one node becoming the input of the next node. This process of passing data from one layer to the next layer is an example of a feedforward artificial neural network. Some embodiments of the inventive concept may provide a rectified linear unit (ReLU) activation function for use at one or more of the neural network nodes.

Returning to FIG. 4, once training is complete, the drug product type model 230 and the drug product integrity model(s) 235 are generated and may operate in inference mode based on a current input feature set for a current drug product. The drug product validation engine 160 may obtain one or more images of a drug product that is held by a drug product packaging system using cameras as described above with respect to FIG. 3. In some embodiments, the drug product images may be pre-processed by one or more operations including, for example, re-sizing the images to normalize the position of the drug product held by the drug product packaging system, i.e., to account for the finger 34 among the multiple fingers 34 that holds the drug product, and identifying the drug product area using, for example, a bounding box. The current input feature set may include information associated with one or more of the plurality of input variables used in the training feature set 205. The current feature set information is embedded into a plurality of input variable vectors using the current feature embeddings module 225, which are then aggregated to provide a drug product input vector. The drug product integrity models 235 may then process the drug product input vector and determine a similarity between the drug product input vector and an intact vector or a defective vector. In some embodiments, this similarity may be based on a cosine similarity score generated based on the drug product input vector and the intact vector and a cosine similarity score generated based on the drug product input vector and the defective vector. Because multiple drug integrity models 235 may be used, aggregation logic 245 may be used to process these intact or defective classifications generated by the various model to reach a final intact or defective prediction 250 for the current drug product. When the drug product is predicted to be intact, the drug product type model 230 may process the drug product input vector and determine a similarity between the drug product input vector and a drug product identification vector corresponding to a drug product name or other identifier, such as NDC, or DIN, for example. Various similarity scores may be generated using, for example, cosine similarity, between the drug product input vector and the numerous vectors corresponding to the drug product names and/or other identifiers, such as NDCs or DINs. The current drug product may be predicted to correspond to the drug product name or other identifier having the highest similarity score. In other embodiments, the drug product input vector may be compared with the drug product identification vector corresponding to the drug product that is expected to be in the package to determine whether there is sufficient similarity therebetween to conclude that the package contains the expected drug product.

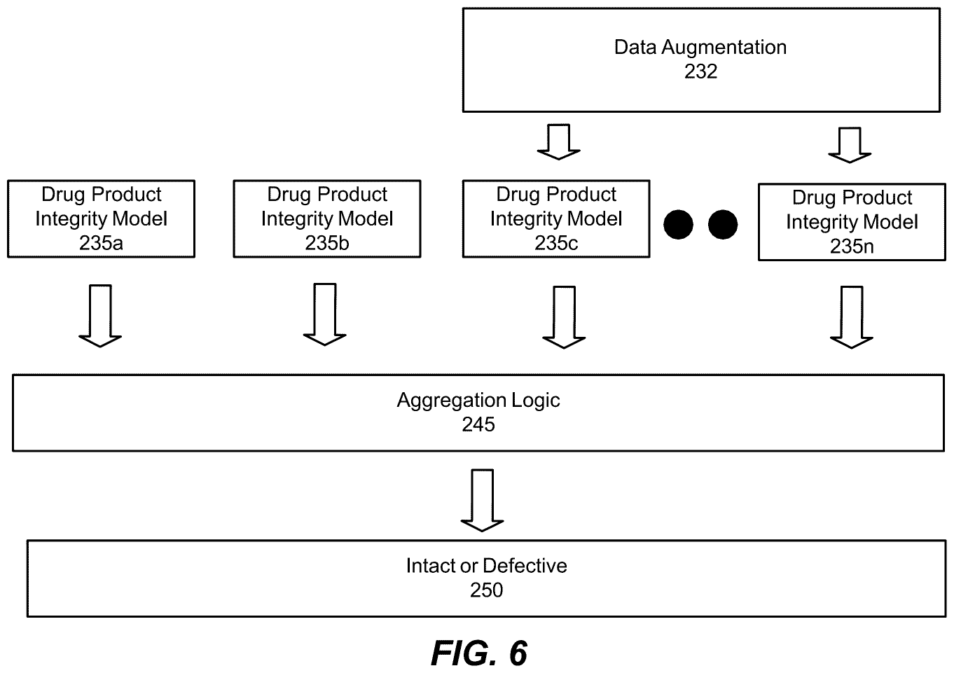

FIG. 6 is a block diagram of the drug product integrity models and aggregation logic of FIG. 4 according to some embodiment of the inventive concept. As shown in FIG. 6, the drug product integrity models 235 may include a plurality of drug product integrity models 235a through 235n. Each of these drug product integrity models 235 may differ from other ones of the drug product integrity models 235 by one or more characteristics. For example, drug product integrity models 235c through 235n may be implemented using a similar neural network platform, but may differ from one another with respect to one or more characteristics of the artificial multi-layer neural network, such as the weights, activation function, threshold, number of layers, and/or number of nodes in the various layers. Moreover, the current feature input set to these drug product integrity models 235c through 235n may be manipulated using the data augmentation module 232 to account for color, orientation, or other variable to better match the data or information for the variables from the training feature set 205. In some embodiments, the data augmentation module 232 may be used to manipulate the current feature set for input to the drug product type model 230 to account for the same variables. The drug product integrity model 235b may differ from other ones of the drug product integrity models 235 in that it is implemented on a different framework or platform. For example, the drug product integrity model 235b may be implemented using the Py Torch framework or platform while the other drug product integrity models 235 are implemented using a different framework or platform. The drug product integrity model 235a may differ from other ones of the drug product integrity models 235 in that it is a neural network configured as a K nearest neighbor neural network model. The K nearest neighbor neural network model determines what the K closest vectors are to the drug product input vector. If, for example, a majority of the K closest vectors correspond to intact drug product, then the model may predict the drug product is intact; otherwise, the model may predict that the drug product is defective. In other embodiments, instead of a majority threshold, a different threshold may be chosen based on whether it is preferable to err on an intact classification or a defective classification. In some embodiments, a triplet loss function may be used to determine the closeness of the drug product input vector to other vectors in the K nearest neighbor neural network model 235a.

The aggregation logic 245 may be used to combine the intact or defective classifications from the plurality of drug product integrity models 235a through 235n to arrive at a final prediction that the drug product matches an intact profile or a defective profile 250. The aggregation logic 245 may combine the classifications in a variety of different ways in accordance with different embodiments of the inventive concept. In some embodiments, each of the classifications of the drug product integrity models 235a through 235n may be given equal weights and the final prediction may be based on a simple majority vote or, as described above, a different threshold other than majority may be chosen based on whether it is preferable to err on an intact classification or a defective classification. In other embodiments, one or more models may be given additional weight. For example, the drug product may be predicted to be intact if a number of the K nearest neighbors of the drug product integrity model 235a that are intact exceeds a threshold and the number of drug product integrity models 235b through 235n predicting the drug product is intact exceeds a threshold. The predictions output from the various drug product integrity models 235a through 235n may also be weighted based on their accuracy over time.

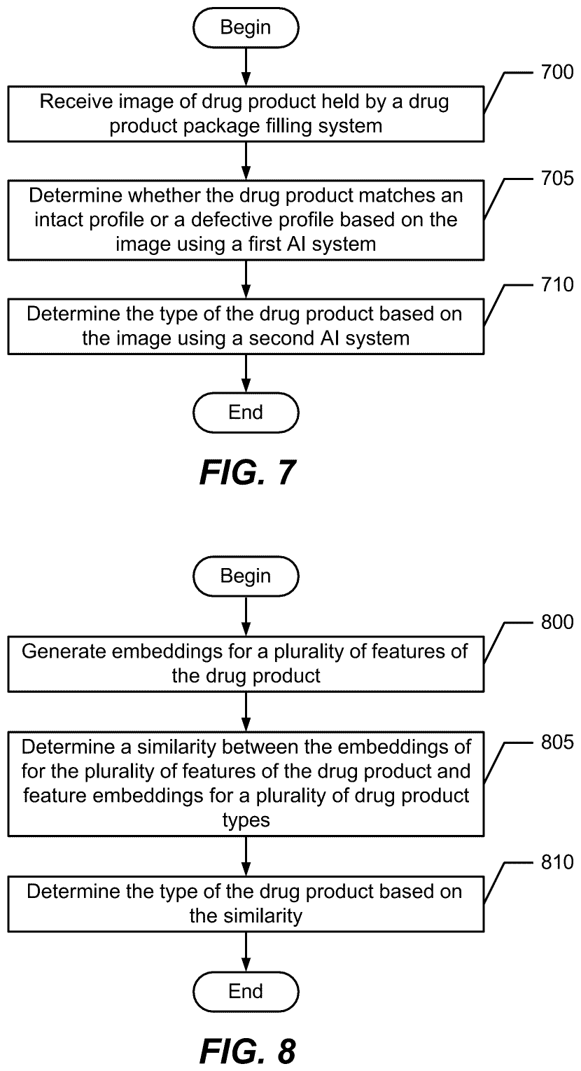

FIGS. 7-9 are flowcharts that illustrate operations for validating drug product held by a drug product packaging system prior to packaging according to some embodiments of the inventive concept. Referring to FIG. 7, operations begin at block 700 where an image of a drug product held by a drug product package filling system or drug product packaging system is received. A determination is made whether the drug product matches an intact profile or a defective profile based on the image using a first AI system. When the drug product is predicted to match an intact profile, then a determination is made of the type of the drug product, e.g., an identification by drug product name or other identifier, such as NDCs or DINs, based on the image at block 710. In other embodiments, the operations of block 710 may not be performed when it is only desired to validate the integrity of the drug product in the package.

Referring now to FIG. 8, operations for determining the type of the drug product begin at block 800 where embeddings are generated for a plurality of features of the drug product. A similarity is determined between the embeddings for the plurality of features of the drug product and feature embeddings for a plurality of drug product types, i.e., drug product names or other identifiers, such as NDCs or DINs, at block 805. The type of the drug product is then determined based on the similarity between the drug product feature embeddings and the drug product type feature embeddings at block 810.

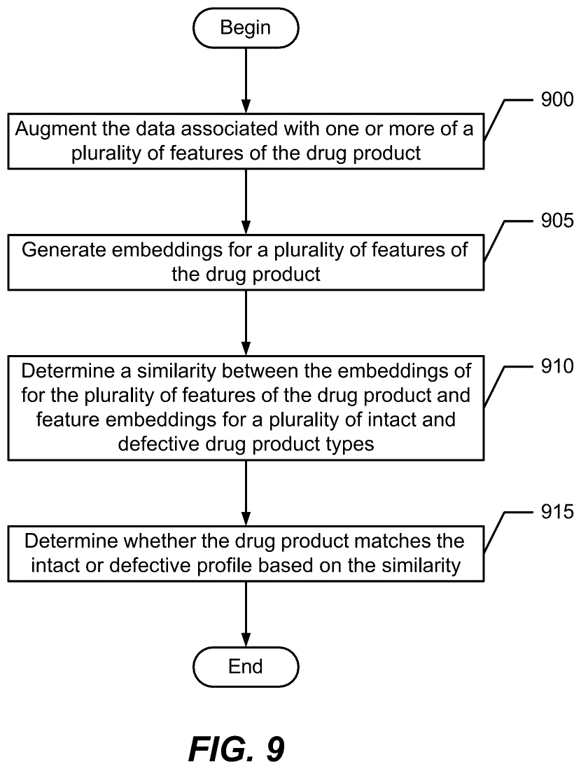

Referring now to FIG. 9, operations for determining whether the drug product matches an intact or defective profile begin at block 900 where the data associated with one or more of a plurality of features may be augmented using, for example, the data augmentation module 232 of FIG. 6, for input to one or more drug product integrity models. Embeddings are generated for a plurality of features of the drug product at block 905 and may be provided as input to one or more drug product integrity models. Each of the drug product integrity models may determine a similarity between the drug product feature embeddings and feature embeddings for a plurality of intact and defective drug product types at block 910. Each of the drug product integrity models may then determine at block 915 whether the drug product matches the intact or defective profile based on the similarity between the drug product feature embeddings and the feature embeddings for the intact and defective drug product types. As described above with respect to FIG. 6, when multiple drug product integrity models are used, the classifications of each of the drug product integrity models may be aggregated using the aggregation logic 245.

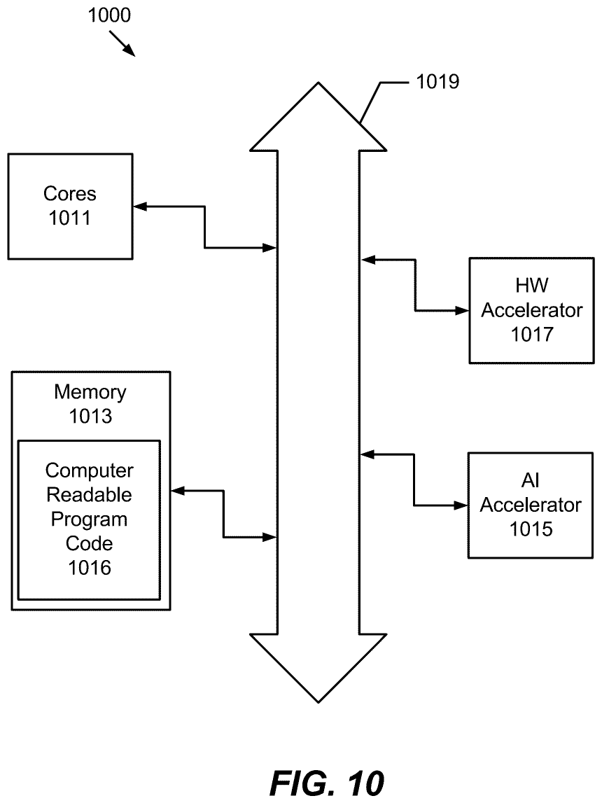

FIG. 10 is a block diagram of a data processing system 1000 that may be used to implement the validation system server 155 of FIG. 1, in accordance with some embodiments of the inventive concept. As shown in FIG. 10, the data processing system 1000 may include at least one core 1011, a memory 1013, an Artificial Intelligence (AI) accelerator 1015, and a hardware (HW) accelerator 1017. The at least one core 1011, the memory 1013, the AI accelerator 1015, and the HW accelerator 1017 may communicate with each other through a bus 1019.

The at least one core 1011 may be configured to execute computer program instructions. For example, the at least one core 1011 may execute an operating system and/or applications represented by the computer readable program code 1016 stored in the memory 1013. In some embodiments, the at least one core 1011 may be configured to instruct the AI accelerator 1015 and/or the HW accelerator 1017 to perform operations by executing the instructions and obtain results of the operations from the AI accelerator 1015 and/or the HW accelerator 1017. In some embodiments, the at least one core 1011 may be an Application Specific Instruction Set Processor (ASIP) customized for specific purposes and support a dedicated instruction set.

The memory 1013 may have an arbitrary structure configured to store data. For example, the memory 1013 may include a volatile memory device, such as dynamic random-access memory (DRAM) and static RAM (SRAM), or include a non-volatile memory device, such as flash memory and resistive RAM (RRAM). The at least one core 1011, the AI accelerator 1015, and the HW accelerator 1017 may store data in the memory 1013 or read data from the memory 1013 through the bus 1019.

The AI accelerator 1015 may refer to hardware designed for AI applications. In some embodiments, the AI accelerator 1015 may include drug product validation functionality configured to provide a service for validating drug product prior to packaging while the drug product is held by the drug product packaging system. The AI accelerator 1015 may generate output data by processing input data provided from the at least one core 1011 and/or the HW accelerator 1017 and provide the output data to the at least one core 1011 and/or the HW accelerator 1017. In some embodiments, the AI accelerator 1015 may be programmable and be programmed by the at least one core 1011 and/or the HW accelerator 1017. The HW accelerator 1017 may include hardware designed to perform specific operations at high speed. The HW accelerator 1017 may be programmable and be programmed by the at least one core 1011.

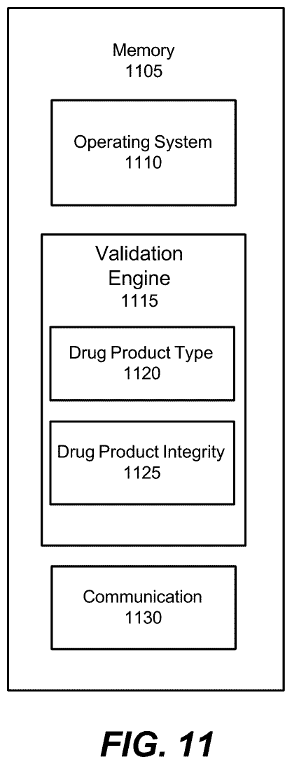

FIG. 11 illustrates a memory 1105 that may be used in embodiments of data processing systems, such as the validation system server 155 of FIG. 1 and the data processing system 1000 of FIG. 10, respectively, to facilitate operation of a system for validating a drug product prior to packaging while the drug product is held by the drug product packaging system. The memory 1105 is representative of the one or more memory devices containing the software and data used for facilitating operations of the validation system server 155 as described herein. The memory 1105 may include, but is not limited to, the following types of devices: cache, ROM, PROM, EPROM, EEPROM, flash, SRAM, and DRAM. As shown in FIG. 11, the memory 1105 may contain three or more categories of software and/or data: an operating system 1110, a validation engine module 1115, and a communication module 1130. In particular, the operating system 1110 may manage the data processing system's software and/or hardware resources and may coordinate execution of programs by the processor.

The validation engine 1115 includes a drug product type module 1120 and a drug product integrity module 1125. The drug product type module 1120 may be configured to perform one or more of the operations described above with respect to the drug product type model 230 of FIG. 4 and the flowcharts of FIGS. 7-9. The drug product integrity module 1125 may be configured to perform one or more of the operations described above with respect to the drug product integrity models 235 of FIGS. 4 and 6 and the flowcharts of FIGS. 7-9. The communication module 1130 may be configured to facilitate communication between the validation system server 155 of FIG. 1 and entities, such as pharmacists, health care professionals, or other entities that may operate or use a drug product packaging system.

Although FIGS. 10 and 11 illustrate hardware/software architectures that may be used in data processing systems, such as the validation system server 155 of FIG. 1 and the data processing system 1000 of FIG. 10, respectively, in accordance with some embodiments of the inventive concept, it will be understood that the present invention is not limited to such a configuration but is intended to encompass any configuration capable of carrying out operations described herein.

Computer program code for carrying out operations of data processing systems discussed above with respect to FIGS. 1-11 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the validation system server 155 of FIG. 1 and the data processing system 1000 of FIG. 10 may each be implemented as a single processor system, a multi-processor system, a multi-core processor system, a virtual computing system, or even a network of stand-alone computer systems, in accordance with various embodiments of the inventive concept. Each of these processor/computer systems may be referred to as a “processor” or “data processing system.”

The data processing apparatus described herein with respect to FIGS. 1-10 may be used to facilitate operation of a drug product validation system according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 1105 when coupled to a processor includes computer readable program code that, when executed by the processor, causes the processor to perform operations including one or more of the operations described herein with respect to FIGS. 1-9.

As described above, embodiments of the inventive concept may provide an AI assisted drug product validation system that may use AI technology, such as multiple artificial neural networks, to validate drug product prior to packaging while the drug product is held by the drug product packaging system. The drug product validation system incorporates one or more neural networks to predict whether the drug product is intact or whether the drug product is defective in some way prior to the drug product being placed in a package. The drug product validation system may also predict a likely drug product name or other identifier for the drug product to ensure the correct drug product is being packaged. By performing this validation while the drug product is still held by the drug product packaging system, packaging waste can be reduced due to defective or incorrectly packaged drug products.

### Further Definitions and Embodiments

In the above-description of various embodiments of the present disclosure, aspects of the present disclosure may be illustrated and described herein in any of a number of patentable classes or contexts including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods, and computer program products according to various aspects of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the disclosure. As used herein, the singular forms “a”, “an” and “the” are intended to include the plural forms as well, unless the context clearly indicates otherwise. It will be further understood that the terms “comprises,” “comprising,” “include”, “including”, “includes”, “have”, “has”, “having”, or variants thereof when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

It will also be understood that, although the terms first, second, etc. may be used herein to describe various elements, these elements should not be limited by these terms. These terms are only used to distinguish one element from another.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

What is claimed is:

1. A method comprising:
receiving an image of a drug product held by a drug product package filling system;
determining whether the drug product matches an intact profile or a defective profile based on the image using a first Artificial Intelligence (AI) system; and
determining, when the drug product matches the intact profile, a type of the drug product based on the image using a second AI system.

2. The method of claim 1, further comprising normalizing the image to account for the position of the drug product on the drug product package filling system.

3. The method of claim 2, wherein the drug product package filling system comprises a plurality of fingers and the drug product is held on one of the plurality of fingers.

4. The method of claim 3, wherein the drug product is held on one of the plurality of fingers using suction.

5. The method of claim 2, wherein determining whether the drug product matches the intact profile or the defective profile based on the image comprises determining whether the drug product matches the intact profile or the defective profile based on the normalized image using the first AI system; and
wherein determining the type of the drug product based on the image comprises determining the type of the drug product based on the normalized image using the second AI system.

6. The method of claim 5, further comprising:
generating embeddings for a plurality of features of the drug product, respectively, using the second AI system; and
determining a similarity between the embeddings for the plurality of features of the drug product and feature embeddings for a plurality of drug product types;
wherein determining the type of the drug product comprises determining the type of the drug product based on the similarity between the embeddings for the plurality of features of the drug product and the feature embeddings for the plurality of drug product types.

7. The method of claim 6, wherein the plurality of features of the drug product comprise drug product shape, drug product size, drug product color, an etching on the drug product, an imprint on the drug product, an area of the drug product, and/or a label on the drug product.

8. The method of claim 6, wherein the plurality of drug product types comprises a plurality of drug product names and/or a plurality of National Drug Code (NDC) identifiers.

9. The method of claim 5, further comprising:
augmenting data associated with one or more of a plurality of features of the drug product; and
generating embeddings for the plurality of features of the drug product, respectively, using the first AI system responsive to augmenting the data associated with the one or more of the plurality of features of the drug product.

10. The method of claim 9, wherein the plurality of features of the drug product comprises drug product shape, drug product size, drug product color, an etching on the drug product, an imprint on the drug product, an area of the drug product, a label on the drug product, cracks in the drug product, uneven surfaces of the drug product, chips in the drug product surface, color deviations in the drug product, shape deviations in the drug product, lamination of the drug product, irregular edges of the drug product, dents in the drug product, splits in the drug product, joints in the seams in the drug product, residue on the drug product, deformation of the drug product, and/or bubbles inside the drug product;
wherein the first AI system comprises a plurality of neural network models, the plurality of neural network models differing from each other with respect to node weights and/or activation functions; and
wherein the method further comprises:
determining, using each of the plurality of neural network models, a similarity between the embeddings for the plurality of features of the drug product and feature embeddings for a plurality of intact and defective drug product types, respectively;
wherein determining whether the drug product matches the intact profile or the defective profile comprises determining, using each of the plurality of neural network models, whether the drug product matches the intact profile or the defective profile based on the similarity between the embeddings for the plurality of features of the drug product and feature embeddings for a plurality of intact and defective drug product types, respectively.

11. The method of claim 10, wherein at least one of the plurality of neural network models comprises an AI framework different than others of the plurality of neural network models.

12. The method of claim 10, wherein the first AI system further comprises a K nearest neighbor neural network model;
wherein the method further comprises:
generating embeddings for a plurality of features of the drug product, respectively, using the K nearest neighbor neural network model; and
determining a similarity between the embeddings for the plurality of features of the drug product and the feature embeddings for the plurality of intact and defective drug product types, respectively;
wherein determining whether the drug product matches the intact profile or the defective profile comprises determining whether the drug product matches the intact profile or the defective profile based on a number of the K most similar feature embeddings of the plurality of intact and defective drug product types that are intact and a number of the K most similar feature embeddings of the plurality of intact and defective drug product types that are defective.

13. The method of claim 12, further comprising:
aggregating the determinations of the plurality of neural network models and the K nearest neighbor network model on whether the drug product matches the intact profile or the defective profile; and
determining whether the drug product matches the intact profile or the defective profile based on the aggregation of the determinations of the plurality of neural network models and the K nearest neighbor network model.

14. A system, comprising:
a processor; and
a memory coupled to the processor and comprising computer readable program code embodied in the memory that is executable by the processor to perform operations comprising:
receiving an image of a drug product held by a drug product package filling system;
determining whether the drug product matches an intact profile or a defective profile based on the image using a first Artificial Intelligence (AI) system; and
determining, when the drug product matches the intact profile, a type of the drug product based on the image using a second AI system.

15. The system of claim 14, wherein the operations further comprise:
normalizing the image to account for the position of the drug product on the drug product package filling system;
wherein determining whether the drug product matches the intact profile or the defective profile based on the image comprises determining whether the drug product matches the intact profile or the defective profile based on the normalized image using the first AI system; and
wherein determining the type of the drug product based on the image comprises determining the type of the drug product based on the normalized image using the second AI system.

16. The system of claim 15, wherein the operations further comprise:
generating embeddings for a plurality of features of the drug product, respectively, using the second AI system; and
determining a similarity between the embeddings for the plurality of features of the drug product and feature embeddings for a plurality of drug product types;
wherein determining the type of the drug product comprises determining the type of the drug product based on the similarity between the embeddings for the plurality of features of the drug product and the feature embeddings for the plurality of drug product types.

17. The system of claim 15, wherein the operations further comprise:
augmenting data associated with one or more of a plurality of features of the drug product; and
generating embeddings for the plurality of features of the drug product, respectively, using the first AI system responsive to augmenting the data associated with the one or more of the plurality of features of the drug product.

18. The system of claim 17, wherein the plurality of features of the drug product comprises drug product shape, drug product size, drug product color, an etching on the drug product, an imprint on the drug product, an area of the drug product, a label on the drug product, cracks in the drug product, uneven surfaces of the drug product, chips in the drug product surface, color deviations in the drug product, shape deviations in the drug product, lamination of the drug product, irregular edges of the drug product, dents in the drug product, splits in the drug product, joints in the seams in the drug product, residue on the drug product, deformation of the drug product, and/or bubbles inside the drug product;
wherein the first AI system comprises a plurality of neural network models, the plurality of neural network models differing from each other with respect to node weights and/or activation functions; and
wherein the operations further comprise:
determining, using each of the plurality of neural network models, a similarity between the embeddings for the plurality of features of the drug product and feature embeddings for a plurality of intact and defective drug product types, respectively;
wherein determining whether the drug product matches the intact profile or the defective profile comprises determining, using each of the plurality of neural network models, whether the drug product matches the intact profile or the defective profile based on the similarity between the embeddings for the plurality of features of the drug product and feature embeddings for a plurality of intact and defective drug product types, respectively.

19. The system of claim 18, wherein the first AI system further comprises a K nearest neighbor neural network model;
wherein the method further comprises:
generating embeddings for a plurality of features of the drug product, respectively, using the K nearest neighbor neural network model; and
determining a similarity between the embeddings for the plurality of features of the drug product and the feature embeddings for the plurality of intact and defective drug product types, respectively;
wherein determining whether the drug product matches the intact profile or the defective profile comprises determining whether the drug product matches the intact profile or the defective profile based on a number of the K most similar feature embeddings of the plurality of intact and defective drug product types that are intact and a number of the K most similar feature embeddings of the plurality of intact and defective drug product types that are defective.

20. A computer program product, comprising:
a non-transitory computer readable storage medium comprising computer readable program code embodied in the medium that is executable by a processor to perform operations comprising:
receiving an image of a drug product held by a drug product package filling system;
determining whether the drug product matches an intact profile or a defective profile based on the image using a first Artificial Intelligence (AI) system; and
determining, when the drug product matches the intact profile, a type of the drug product based on the image using a second AI system.
